(19) United States
(12) Patent Application Publication
(10) Pub. No.: US 2025/0268646 A1
(43) Pub. Date: Aug. 28, 2025

(54) ASSEMBLIES FOR POSITIONING INSTRUMENTS FOR PERCUTANEOUS PROCEDURES

(71) Applicant: André FAURE, Sarasota, FL (US)

(72) Inventor: André FAURE, Sarasota, FL (US)

(21) Appl. No.: 18/858,628

(22) PCT Filed: May 2, 2023
(86) PCT No.: PCT/EP2023/061570

§ 371 Date: Oct. 21, 2024

(30) Foreign Application Priority Data
May 3, 2022 (EP) 22171479.3

(51) Int. Cl. A61B 18/14; A61B 18/00; A61B 90/00

(52) U.S. Cl. CPC A61B 18/14; A61B 90/08; A61B 2018/00577; A61B 2018/1425; A61B 2018/1435

(57) ABSTRACT

An assembly is disclosed for positioning elongate instruments for percutaneous procedures. The assembly has a first guiding member with a first body, a first guiding hole extending through the first body for receiving a coiled instrument, the first guiding hole having a first axis. The assembly further includes a second guiding hole, wherein the second guiding hole is coaxial with the first guiding hole and has an essentially cylindrical shape. The first guiding hole extends at a periphery of the second guiding hole. A second guiding member has a second body with opposite first and second main surfaces, a third guiding hole having an essentially cylindrical shape and extending through the second body. The second guiding member is attached or attachable to a periphery of the first guiding member such that the third guiding hole has an axis substantially parallel to the first axis.

### TECHNICAL FIELD

The present disclosure is related to assemblies for positioning various instruments, such as coiled electrodes and various types of probes with respect to an organ in percutaneous procedures. Particularly, the present disclosure is related to an assembly for positioning electrodes for radiofrequency ablation and to procedures utilizing such assembly, more particularly for prostate procedures.

### BACKGROUND ART

Since the implantation of radioactive seeds in 1980s, transperineal procedures for treating different conditions or realizing biopsies in the prostate became more and more popular. A device is typically inserted in the prostate through the perineum under real time transrectal ultrasound (TRUS) visualization. Several prostate navigation systems have been described, sometimes associated with computer software, using different technologies such as mechanical, magnetic or visual tracking. The major drawback of all those systems is they basically depend on a fixed reference point (with a physical attachment to a surgical table, stepper or ultrasound probe).

The main challenge of these navigation and positioning systems is that the prostate is highly mobile and its shape can change during a given procedure because of increase or decrease in blood flow. This makes any fixed reference point guiding or positioning difficult and imprecise.

WO 2017/093325 describes an assembly for positioning electrodes for radiofrequency ablation comprising a first linear motion guide defining a first axis of motion with a first carriage operable to move along the first axis of motion and an XY positioner operable to move a second carriage in a two-dimensional space perpendicular to the first axis of motion. An imaging device, such as a TRUS probe, is mounted on the first carriage. An electrode guiding device is mounted to the second carriage. A second linear motion guide configured to move a third carriage along an axis parallel to an axis of motion of the second carriage allows for positioning a template for inserting tissue stabilising instruments independently of the electrode guiding device. One drawback of the above system is that it is cumbersome resulting in increased complexity and cost. In addition, since the template and the electrode guiding device are positioned independently, it results to be difficult to calculate a correct position for the electrode guiding device from a known position of the template.

### SUMMARY

There is a need in the art to provide assemblies allowing for improved, more accurate and/or easier positioning of instruments with regard to a body organ. There is a need in the art to provide such assemblies which are of simple construction and easy to use. There is a need in the art to provide such assemblies allowing to easily relate positions of different inserted instruments with one another.

According to a first aspect of the disclosure, there is therefore provided an assembly comprising a first guiding member and a second guiding member. The first guiding member comprises a first body and a first guiding hole extending through the first body. A second guiding hole extends coaxially with the first guiding hole and is surrounded by the first guiding hole. The second guiding hole advantageously has an essentially cylindrical shape. The first body and advantageously the second guiding hole extend between a first end and a second end opposite the first end. The second guiding member comprises a second body. At least one, advantageously a plurality of third guiding holes extend through the second body.

The second guiding member is attached or attachable to the first guiding member at a periphery of the first guiding member such that the one or more third guiding holes have axes substantially parallel to an axis of the first guiding hole. A connector system is advantageously configured to possibly releasably attach the second guiding member to the first guiding member. The connector system can comprise a recess, comprising or consisting of a through hole, provided in the second guiding member, such as through the second body. The recess is configured to receive the first body, e.g. an external surface portion of the first body mates with the recess. The second body extends at a periphery of the first body when attached.

With such an assembly the position of the third guiding holes can be easily made to relate to the position of the first and second guiding holes. When inserting surgical instruments through these guiding holes and verifying their position through known medical imaging techniques, such as ultrasound, it is easy to obtain an insertion location for a surgical instrument that corresponds with a desired internal location in the patient. This is achieved because, firstly, any instrument inserted through a guiding hole forms a reference point between the internal organ and the external position of the assembly, and secondly, the positions of the various guiding holes in the assembly can be easily related to one another. It is therefore provided a simple yet efficient percutaneous instrument positioning method allowing the surgeon to save time, reduce the number of erroneous insertions of instruments and obviating the need of expensive and cumbersome positioning systems. Assemblies as described herein are particularly useful for radiofrequency ablation techniques.

The present disclosure also provides methods of positioning instruments within a prostate or other body organ. A method according to aspects of the present disclosure advantageously comprises utilizing the assembly as described herein. The first guiding member is positioned in proximity of a patient, at an external location corresponding with an internal location of an organ or tissue to treat. A first straight needle instrument is inserted through the second guiding hole and percutaneously towards the organ or tissue. The position of the first needle is assessed, e.g. through an imaging system, and a relative position of the internal location with respect to the first needle is determined. A third guiding hole positioned in correspondence with the relative position is determined. A second straight needle instrument is inserted through the third guiding hole and percutaneously to reach the organ or tissue. At least the second straight needle and possibly the first straight needle are kept in the body while the assembly is repositioned. The assembly is repositioned to slide the second guiding hole over the second needle. The third guiding hole can be slid over the first needle for improved stabilization. A further instrument is inserted through the first guiding hole. A surgical procedure can now be started utilizing instruments inserted through the first and/or second guiding holes.

### BRIEF DESCRIPTION OF THE FIGURES

Aspects of the present disclosure will now be described in more detail with reference to the appended drawings, wherein same reference numerals illustrate same features and wherein:

FIG. 1 represents a perspective side view of an electrode guiding member (first guiding member) according to an aspect of the present disclosure;

FIG. 2 represents a top view of the electrode guiding member of FIG. 1;

FIG. 3 represents a perspective view of a template guide member (second guiding member) according to an aspect of the present disclosure;

FIG. 4 represents a perspective view of an assembly of the electrode guiding member of FIG. 1 and the template guide member of FIG. 3;

FIG. 5 represents a perspective view of another embodiment of a template guide member (second guiding member) according to an aspect of the present disclosure;

FIG. 6 represents a perspective view of an assembly of the electrode guiding member of FIG. 1 and the template guide member of FIG. 5;

FIG. 7 represents a perspective side view of another embodiment of an electrode guiding member (first guiding member) according to an aspect of the present disclosure;

FIG. 8 represents a top view of the electrode guiding member of FIG. 7;

FIG. 9 represents a perspective view of the electrode guiding member of FIG. 1 in which an electrode assembly comprising a coiled electrode and a straight (needle) electrode are inserted;

FIG. 10 represents a flow diagram of a method of percutaneous procedures according to the present disclosure;

FIG. 11 represents a diagram of a setup for positioning a prostate ablation instrument according to the present disclosure;

FIG. 12 represents a diagram of a setup for prostate ablation according to the present disclosure.

### DETAILED DESCRIPTION

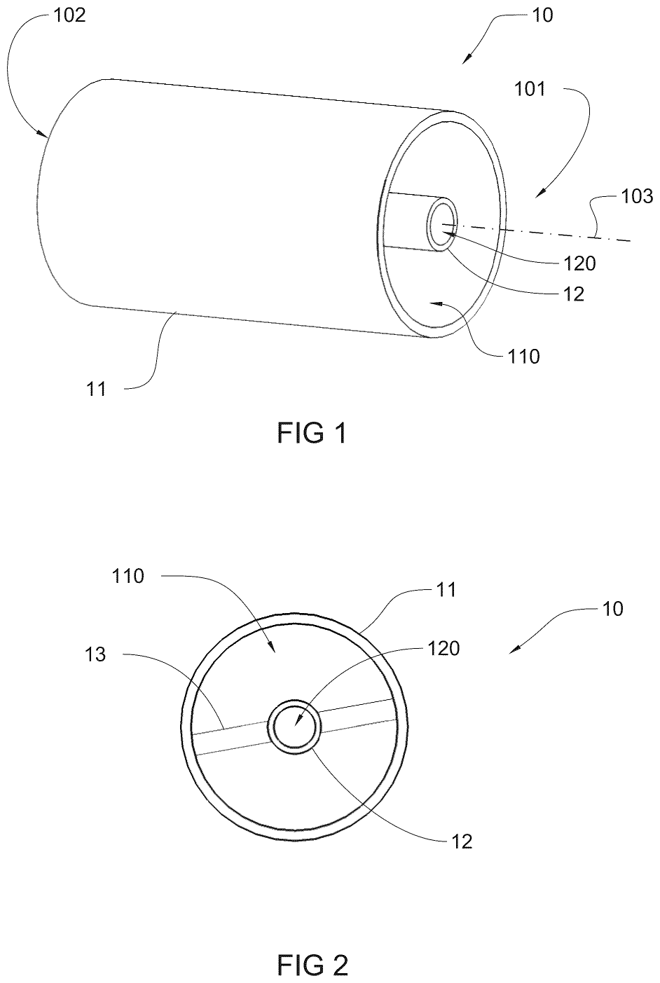

Referring to FIGS. 1-2, a first guiding member 10, referred to herein as electrode guiding member, comprises a first body 11, which can be essentially tubular, extending between a first end 101 and a second, opposite end 102, along an axis 103. The first body 11 defines a through hole 110 which can be essentially cylindrical with axis 103 defining the cylinder axis. Inside through hole 110 is arranged a second body 12 which can be essentially tubular. Second body 12 extends from the first end 101 to the second end 102 over a length which can be identical to, or different from, a length of the first body 11, along axis 103. Second body 12 defines a through hole 120 which is essentially cylindrical. Through holes 110 and 120 are coaxial on axis 103. Through hole 120 (second body 12) can be longer, shorter or of same length compared to through hole 110 (first body 11), along axis 103.

An external diameter of the second body 12 is smaller than a diameter of through hole 110, such that body 12 can be completely accommodated in through hole 110. Through hole 120 has a smaller diameter compared to the diameter of through hole 110. The second body 12 can be attached to the body 11 in any suitable way, such as by stalk-like connecting members 13 extending transversely through the through hole 110. The first and the second body can hence be formed as one integral body, e.g. as made by moulding, by 3D printing or other layered manufacturing techniques. Alternatively, the first and the second body can be assembled from separate parts.

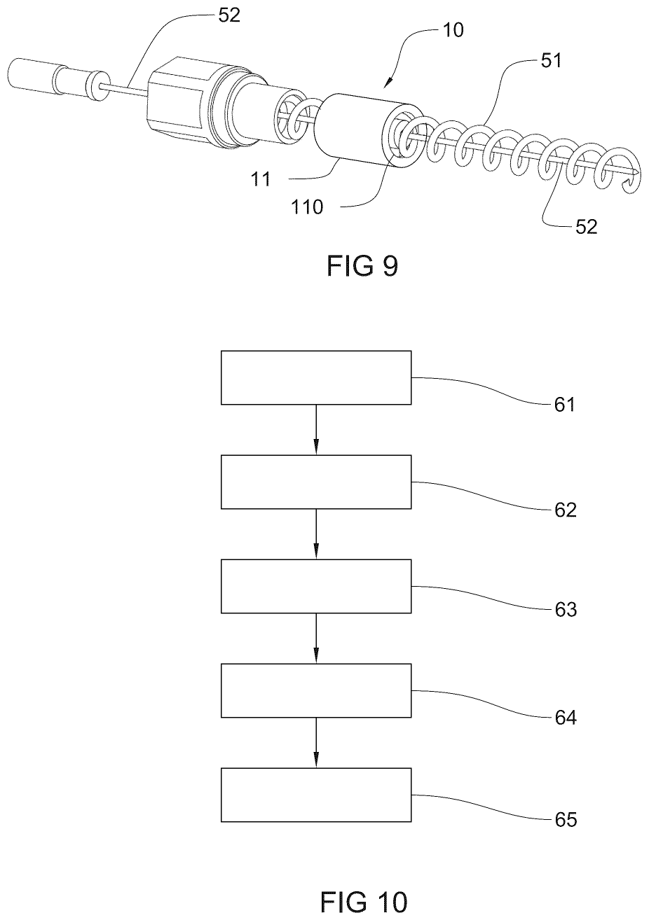

As illustrated in FIG. 9, through hole 110 is advantageously configured to guide a coiled electrode 51. The diameter of through hole 110, which can be cylindrical, advantageously mates with an outer diameter of coiled electrode 51. By so doing, it is effectively prevented that the coils of coiled electrode 51 would expand radially when coiled electrode 51 is inserted through the skin and/or when the coiled electrode 51 engages the organ or other tissue. Coiled electrode 51 is received in through hole 110 such that it can advance or be retracted with respect to the electrode guiding member 10, e.g. by sliding and/or rotational motion. It will be appreciated that through hole 110 can have suitable shapes other than cylindrical, such as helical, particularly having a pitch mating with a pitch of a helix of the coiled electrode 51.

Through hole 120 is configured to receive a straight instrument, such as a straight electrode 52 and therefore is advantageously of cylindrical shape. A diameter of through hole 120 is configured to mate with a diameter of straight electrode 52. Electrodes 51 and 52 can be configured to be operated as bipolar electrodes for ablation of tissue by radiofrequency or microwave electrical power as known in the art. Electrode guiding member 10 advantageously ensures a correct positioning of the electrodes 51 and 52, which can be arranged coaxially.

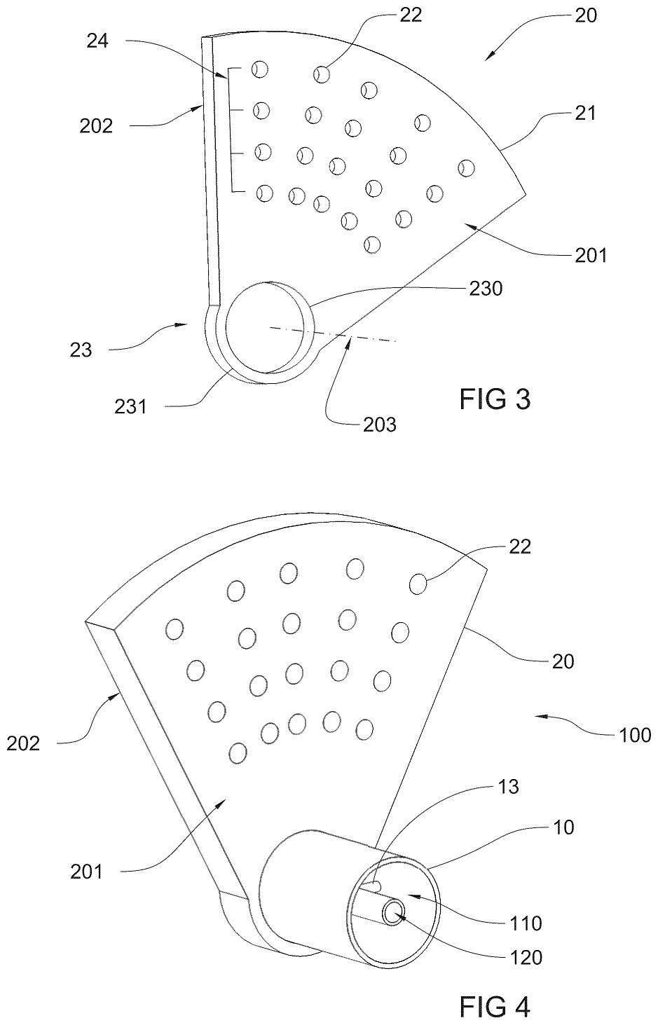

Referring to FIGS. 3-4, a second guiding member 20, referred to herein as template member, comprises a body 21 having a first main surface 201 and opposite second main surface 202. First and second main surfaces 201, 202 can be planar, and body 21 can be plate-shaped. Body 21 is substantially pie-shaped, extending radially from an axis 203, over a specified angle about axis 203. First and/or second main surfaces 201, 202 can be perpendicular to axis 203. A plurality of through holes 22 are arranged through body 21, extending from the first main surface 201 to the second main surface 202. Through holes 22 have axes advantageously parallel to axis 203. Through holes 22 can be ordered in any convenient pattern, e.g. arranged in one or more single row arrays extending along radial directions from axis 203, each array having a different angular orientation about axis 203.

Through holes 22 advantageously have a same diameter as the diameter of through hole 120 of electrode guiding member 10, and are advantageously of cylindrical shape. Through holes 22 are advantageously configured to (slidingly) receive a straight instrument, such as a biopsy needle, or straight electrode 52. Through holes 22 can be configured to receive any other surgical or therapeutic instrument for percutaneous use, such as probes or needles for cryoablation, microwave, radiofrequency, radioactive seeds and so on.

A distance marking 24 can be provided on the first main surface 201, in registration with the through holes 22. Marking 24 advantageously allows to determine a distance of each through hole 22 from a predetermined reference, such as axis 203.

The template member 20 can be provided with a connector system 23 for coupling to the electrode guiding member 10 so as to obtain an assembly 100. Connector system 23 can allow for a releasable connection between the electrode guiding member 10 and the template member 20. Advantageously, the connector system 23 is configured to position (secure) template member 20 relative to electrode guiding member 10 such that axes 103 and 203 coincide. Any suitable connector system can be utilized, e.g. a snap-fit connector, or a threaded connector, and the connector system can comprise co-operating connector parts arranged on both the electrode guiding member and the template member. It will be appreciated that the electrode guiding member and the template member can be formed integrally in the alternative.

In some examples, connector system 23 comprises a through hole 230 arranged in an annular portion 231 of body 21, which through hole 230 mates with body 11 of electrode guiding member 10. Through hole 230 can have axis 203 as its centre. At least part of the outer surface of body 11 can be configured to snugly fit in through hole 230. While through hole 230 and the outer surface of body 11 are shown as cylindrical in the figures, it will be appreciated that other shapes, such as polygonal, are possible. In some examples, connector system 23 is configured to allow template member 20 to rotate about the common axes 103-203 with respect to the electrode guiding member 10. This provides a higher flexibility in positioning the template member relative to the patient and the body organ.

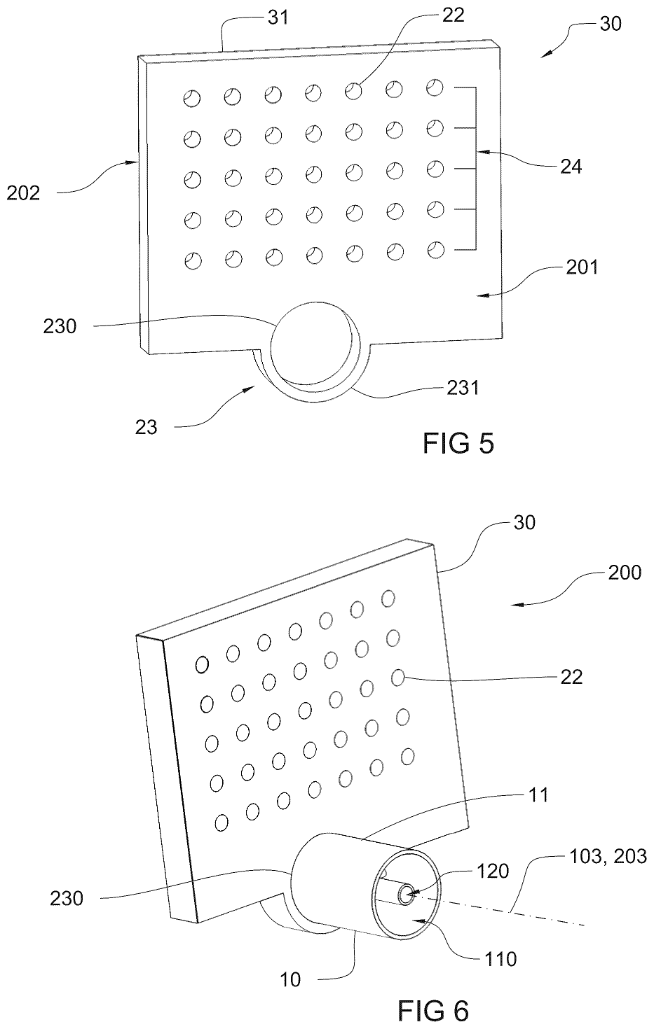

Referring to FIGS. 5-6, another embodiment of a template member 30 can be mounted on electrode guiding member 10 to obtain assembly 200. Template member 30 differs from template member 20 in the disposition of the through holes 22. The shape of body 31 of template member 30 is advantageously substantially rectangular rather than pie shaped like body 21 of template member 20. The through holes 22 of body 31 are arranged in a rectangular pattern with a plurality of single row arrays of through holes 22 arranged in parallel. Template member 30 can have a same distance marker 24 and/or connector system 23 as template member 20.

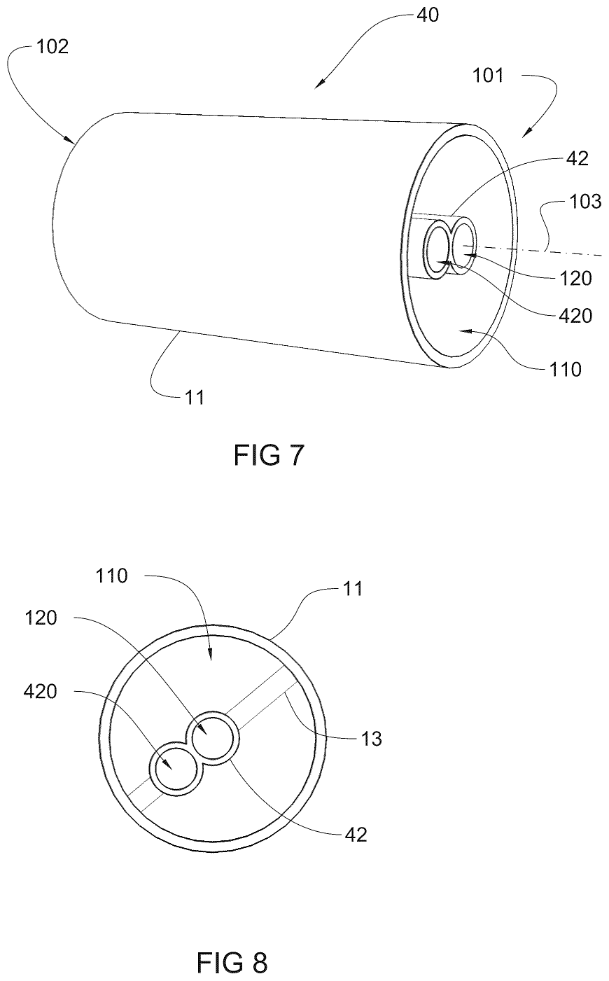

Referring to FIGS. 7-8, another embodiment of the electrode guiding member 40 differs from electrode guiding member 10 in that the second body 42 comprises, besides through hole 120 which is coaxial with through hole 110, one or more further through holes 420. Through hole 420 is adjacent to through hole 120 and advantageously extends parallel to through hole 120 but is eccentric to through holes 120 and 110 (i.e., eccentric to axis 103). Still, through hole 420 is advantageously completely comprised in, and surrounded by, through hole 110. Through holes 420 and 120 advantageously have same diameter and both can be cylindrical. The first body 11 and second body 42 can be likewise connected through connecting members 13, and they may be integrally formed. In some examples, the second body 42 is formed such that it allows the coiled electrode 51 to be inserted in through hole 110 and the straight electrode 52 to be inserted in one of through hole 120 and through hole 420, as needed, and be guided by it, from the first end 101 to the second end 102 and vice versa. Through hole 420 allows to arrange the straight electrode 52 (FIG. 9) eccentric with respect to coiled electrode 51.

The template member and the electrode guiding member can be made of a polymer, metal or any other biocompatible material.

The assemblies 100, 200 are advantageously utilized in various percutaneous procedures, particularly transperineal procedures, more particularly in prostate procedures. A particular example is now described with respect to prostate ablation; it will however be appreciated that a same flow of operation can be applied in other transcutaneous procedures.

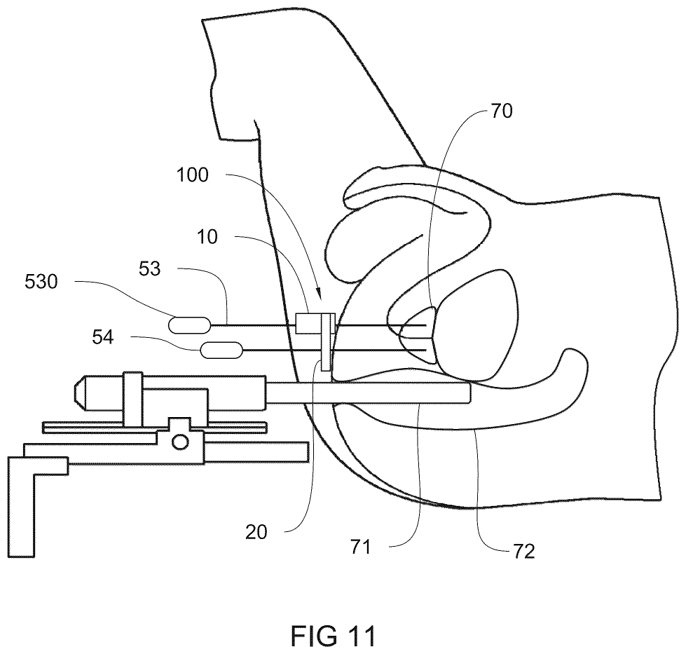

Referring to FIGS. 10-11, in a first operation 61, the whole assembly 100, 200, or only the electrode guiding member 10, 40 is placed against the perineum. The electrode guiding member can be held manually by the surgeon, in a random position close to an imaging probe 71, such as a TRUS probe that may be prior inserted into the patients' rectum 72. Alternatively, the assembly or electrode guiding member can be attached to a positioning table (not shown) and held initially in place by it.

A straight needle instrument 53, particularly a stabilization needle, is inserted in through hole 120 and further into the patient, advantageously penetrating the prostate gland 70. The insertion operation can be monitored through the imaging probe 71. This needle 53 serves as an initial reference for determining the correct final position of an electrode assembly for ablation. The needle 53 keeps a fixed reference with respect to the prostate gland 70. At this point, the electrode guiding member 10 can be freed from any external positioning system, if any, since the needle 53 keeps a fixed reference with respect to the prostate.

In operation 62, the surgeon will identify the position of the inserted needle 53 within the prostate and define, with the aid of the imaging probe 71 and possibly any kind of navigation software, the relative position from the inserted needle 53 to a desired position of an ablation electrode for an ideal treatment planning.

The template member 20 is attached to the electrode guiding member 10, which is held in place by the inserted needle 53. It is possible that template member 20 and electrode guiding member 20 are attached beforehand. The template member can be rotated on axis 103/203 (e.g. about the inserted needle 53), until a desired orientation is achieved.

In operation 63, the through hole 22 of the template member which corresponds to the desired position is determined, e.g. with the aid of distance marking 24. A second needle 54 is inserted in the identified through hole 22, advantageously to penetrate the prostate gland 70. Insertion of the second needle allows stabilization of the prostate gland 70 with respect to the assembly 100, eliminating problems that prior art systems have in terms of reaching the right spot in a highly mobile organ such as the prostate. The insertion of the second needle 54 is monitored through the visualization probe 71 and its actual position with respect to the prostate can be determined.

The surgeon can repeat operation 62 and/or 63 for placing one or more additional needles in one or more identified through holes 22, particularly if it failed to reach a desired position with the second needle.

If the ideal spot for placing a further needle is out of reach of the through holes 22 of the template member, the surgeon can remove the electrode guiding member 10 from the first needle 53 without however removing the first needle from the prostate. The electrode guiding member is repositioned by sliding through hole 120 over the second needle. Operations 62 and 63 are repeated.

Any one of the needles 53, 54 may be operated as a radiofrequency electrode, just like straight needle electrode 52. To this end, such needles may comprise an electrically conducting body and tip. The body may be coated with an electrically isolating coating, while the tip is exposed. A handle 530 can be removably attached to a proximal end of the needle 53, opposite the tip. The handle 530 may alternatively be dispensed with and a proximal end of the body (opposite the tip) may be exposed for connecting to a radiofrequency power source.

In operation 64, when a needle 54 has been inserted at a desired position in the prostate, the surgeon repositions the electrode guiding member 10 on that needle 54 (by removing it from the needle 53 on which it was positioned previously and sliding through hole 120 over the needle 54). The needle 54 on which the electrode guiding member is newly positioned can be utilized as a straight electrode 52. The needle on which the electrode guiding member was previously positioned can be used for stabilization purposes. In some examples, a through hole 22 of the template member can be slid on this needle when repositioning the electrode guiding member for improved stability. As the assembly 100 is now stabilized with respect to the prostate, the needle 54 can be removed and replaced by any other suitable instrument (e.g. electrode 52, if needle 54 cannot be utilized for such purposes) for performing the ablation procedure.

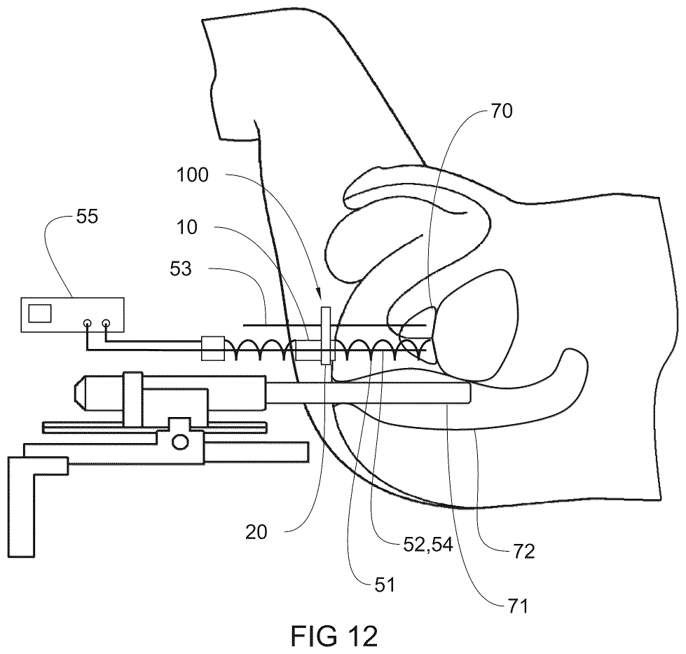

In operation 65, the coiled electrode 51 is inserted in through hole 110 and further transperineally into the prostate to obtain a configuration as shown in FIG. 9 and FIG. 12. An ablation procedure can now be started by connecting the coiled electrode 51 and the straight electrode 52 (or 54) to a radiofrequency power source 55 and operating the electrodes 51, 52.

In an advantageous procedure, when a plurality of areas of the prostate are to be treated, a needle can be inserted in relation to each of the areas to be treated before starting any treatment. This can be performed by utilizing an appropriate template member. This will eliminate issues related to prostate movement or deformation between consecutive treatment sessions. This also eliminates the risk of losing track of treated zones when multiple ablation spots are required.

In some situations, coiled electrodes 51 and needle electrodes 52 can suffer deviation when penetrating through the skin and within the prostate. This causes a risk that the tips of the two electrodes touch each other rendering it impossible to complete an ablation. Removing one needle and inserting again, often turns out to be a useless procedure as the needle tends to create a channel and repeat the same pathway within the prostate on and on. In this case, the electrode guiding member 40 with a through hole 420 adjacent the central through hole 120 allows a more convenient repositioning of the needle 52 avoiding the above problem by inserting needle 52 through through hole 420 rather than through hole 120. It will be appreciated that the electrode guiding member can be rotated on its axis 103 to position the through hole 420 at any desired location.

1. An assembly for positioning elongate instruments for percutaneous procedures, the assembly comprising:
a first guiding member comprising a first body extending between a first end and a second end opposite the first end;
a first guiding hole extending through the first body and configured to receive a coiled instrument, the first guiding hole having a first axis oriented from the first end to the second end;
a second guiding hole extending between the first end and the second end, wherein the second guiding hole is coaxial with the first guiding hole and has a cylindrical shape, wherein the first guiding hole extends at a periphery of the second guiding hole;
a second guiding member comprising a second body having opposite first and second main surfaces; and
at least one third guiding hole having a cylindrical shape and extending through the second body;
wherein the second guiding member is configured to be attached to a periphery of the first guiding member such that the at least one third guiding hole has an axis substantially parallel to the first axis.

2. The assembly of claim 1, comprising a connector system configured to attach the second guiding member to the first guiding member.

3. The assembly of claim 2, wherein the connector system comprises a recess in the second guiding member, wherein the recess is configured to receive the first body.

4. The assembly of claim 3, wherein the first body comprises an external surface portion mating with the recess.

5. The assembly of claim 4, wherein the external surface portion is substantially cylindrical and coaxial with the first guiding hole.

6. The assembly of claim 3, wherein the recess comprises a through hole.

7. The assembly of claim 3, wherein the recess is coaxial with the first guiding hole.

8. The assembly of claim 3, wherein the connector system comprises one or more annular portions surrounding the recess.

9. The assembly of claim 2, wherein the connector system is configured to attach the second guiding member to the first guiding member such that the second guiding member is pivotal on the first axis with respect to the first guiding member.

10. The assembly of claim 1, wherein the first body has a substantially cylindrical external surface.

11. The assembly of claim 1, wherein the first guiding hole is substantially cylindrical.

12. The assembly of claim 1, wherein the first body comprises a first tubular part defining the first guiding hole and a second tubular part defining the second guiding hole, wherein the second tubular part is arranged inside the first guiding hole, wherein the first body comprises one or more connecting members connecting the first tubular part to the second tubular part, and wherein the connecting members extend transversely through the first guiding hole.

13. The assembly of claim 1, wherein the second guiding member comprises a plurality of the third guiding holes arranged in a plurality of single row arrays.

14. Assembly The assembly of claim 13, wherein the single row arrays are disposed in radial directions from the first axis.

15. Assembly The assembly of claim 13, wherein the second guiding member further comprises distance marks for the third guiding holes provided on the second body.

16. The assembly of claim 1, further comprising a fourth guiding hole extending through the first body, wherein the fourth guiding hole is parallel and eccentric with respect to the second guiding hole, and wherein the first guiding hole is arranged at a periphery of the second guiding hole and the fourth guiding hole.

17. The assembly of claim 1, further comprising a first coiled electrode device for radiofrequency ablation and a second needle-shaped electrode device for radiofrequency ablation, wherein a first diameter of the first guiding hole mates with an outer diameter of the first coiled electrode device and a second diameter of the second guiding hole mates with a diameter of the second needle-shaped electrode device.

18. The assembly of claim 1, wherein a diameter of the second guiding hole is substantially identical to a diameter of the at least one third guiding hole.

19. The assembly of claim 13, wherein the single row arrays are disposed parallel to one another.

20. The assembly of claim 16, wherein the fourth guiding hole has substantially a same diameter as a diameter of the second guiding hole.
